(19) United States
(12) Patent Application Publication
(10) Pub. No.: US 2014/0205662 A1
(43) Pub. Date: Jul. 24, 2014

(54) SYSTEM AND METHOD FOR DIFFERENTIALLY TIMED LOCAL DELIVERY OF A COMBINATION PHARMACEUTICAL PREPARATION FOR ORAL THERAPY

(71) Applicants: Steven P. Engebretson, Old Field, NY (US); Nathan Blumberg, Smithtown, NY (US); Lorne Golub, Smithtown, NY (US)

(72) Inventors: Steven P. Engebretson, Old Field, NY (US); Nathan Blumberg, Smithtown, NY (US); Lorne Golub, Smithtown, NY (US)

(73) Assignee: The Research Foundation for The State University of New York, Albany, NY (US)

(21) Appl. No.: 13/883,133

(22) PCT Filed: Nov. 3, 2011
(86) PCT No.: PCT/US11/59048

§ 371 Date: Jul. 30, 2013

Related U.S. Application Data
(60) Provisional application No. 61/410,619, filed on Nov. 5, 2010.

(51) Int. Cl. A61K 9/50; A61K 31/192; A61K 38/18; A61K 31/65; A61K 31/4164; A61K 31/407

(52) U.S. Cl. CPC A61K 9/50; A61K 31/4164; A61K 31/407; A61K 38/1858; A61K 31/65; A61K 31/192
USPC 424/457; 514/8.2; 514/154

(57) ABSTRACT

The present invention is directed to a system and method for treating oral diseases in a subject. This method comprises the following steps; diagnosing the subject, and choosing one or a combination of medications, their dosing and desired time course and then delivering the medications to the desired site in the periodontal pocket by administering a plurality of microcapsules, capsules, the microcapsules comprising one or more pharmaceutical agent(s) that are released in a predetermined manner and in accordance with the pathophysiology of the targeted disease.

### FIELD OF THE INVENTION

The invention relates generally to the field of treating oral diseases by delivery of pharmaceuticals or combinations of pharmaceuticals stored in microcapsules and delivered in a differential temporal manner.

### BACKGROUND OF THE INVENTION

Chronic periodontitis (CP) is a destructive multi-factorial complex microbial disease initiated by Gram-negative microbes. CP involves progressive loss of the alveolar bone around the teeth, and if left untreated, can lead to the loosening and subsequent loss of teeth. CP pathogenesis has both infectious and inflammatory (host response) components, and the successful resolution of CP depends on appropriate wound healing. A diagnosis of periodontitis is established by inspecting the periodontal tissues (gums) around the teeth with a probe and by evaluating a patient's X-ray films.

Typically, for a healthy subject who is not suffering from CP, the healthy subject's gum (gingival) forms a healthy interface with the tooth consisting of an epithelial attachment, periodontal ligament, and alveolar bone. The space between the tooth and the gingiva in health is a shallow space (sulcus). As can be seen in FIG. 1, in a healthy subject, there is a small space between the subject's gum (1) and the subject's tooth (3) referred to as a gingival sulcus (5). The gingival sulcus (5) normally is shallow in a healthy subject. For a subject suffering from CP, the gingival sulcus becomes larger and deeper as the gum detaches from the surface of the tooth, and is then referred to as a periodontal pocket. As can be seen in FIG. 2, the subject suffering from CP has gum (7) tissue that is detached from the tooth (3) forming a periodontal pocket (9). As the CP increases in severity, the gingiva (7) further detaches from the surface of tooth (3), creating a deeper periodontal pocket (9). A subject with a periodontal pocket (9) of about 5 millimeters (mm) or greater, and has lost periodontal attachment tissue of that amount is considered to have severe CP, and is in need of treatment.

Traditionally, CP is initially treated by non-surgical scaling of the teeth below the gumline with or without local anesthesia. If pockets do not heal appropriately or become too deep for non-surgical treatment, surgery with or without antimicrobial therapy is required to treat CP. Periodontal treatments may be effective, but they are invasive, costly, time consuming, and potentially painful.

Non-surgical treatment of periodontitis may be enhanced by placing medication into the periodontal pocket. Such local delivery treatments are useful, but have limitations. For example, minocycline microspheres are currently used as adjunctive treatments to scaling, but have unfavorable release kinetics, and target only one aspect of periodontitis etiology, namely periodontal pathogens (microbes). Further, current antimicrobial delivery technologies have poor release kinetics, and may release up to one half of the dose within 24 hours after placement. Treatments are needed that provide for sustained release of medication(s), in a controlled manner.

Further, what is desired is a therapy for the treatment of periodontitis, guided bone regeneration, guided tissue regeneration, and other oral diseases, that would aid in the healing of oral wounds and diseases. Further, what is desired is a method for delivery of one or multiple drugs, individually or in combination, at varied specified times. Embodiments of the present application provide a system and methods that address the above and other issues.

### SUMMARY OF THE INVENTION

The present invention is directed to a method for treating oral diseases in a subject in need of such treatment and utilizing a combination of a drug delivery system and pharmaceutical agent(s) to implement the method of treatment. The invention is directed towards a method and system whereby a combination of microcapsules containing one or more pharmaceutical agents is used to: 1) treat oral diseases such as periodontitis, peri-implantitis, sinus infections, mucositis, dental carries, gingivitis and halitosis; 2) aid in recovery after tooth extraction; 3) aid in bone regeneration, tissue regeneration, or both; 4) aid in sinus augmentation and/or 5) treat other suitable oral diseases. This method comprises the following steps; diagnosing the subject, choosing a course of treatment with pharmaceutical agents, administering a plurality of microcapsules, the microcapsules containing one or more pharmaceutical agents to facilitate disease resolution, the release of agents corresponding to the pathophysiology of the disease.

### BRIEF DESCRIPTION OF THE DRAWINGS

The present invention will be better understood by reference to the following drawings of which:

FIG. 1 is a representative view of a healthy subject's tooth and gum;

FIG. 2 is a representative view of a subject's tooth and gum who is suffering from periodontal disease;

FIG. 3 is a representative view of microcapsules;

FIG. 4 is a representative view of microcapsules

FIG. 5 is a representative view of microcapsules;

FIG. 6 is a representative view of a plurality of microcapsules containing different agents, with each microcapsule being diagrammatically represented by an oval;

FIG. 7 is a representative view of a subject's tooth and gum who is suffering from periodontal disease after administration of a dispersed plurality of microcapsules;

FIG. 8 is a graphical view of the cytotoxicity of doxycycline;

FIG. 9 is a graphical view of the cytotoxicity of flurbiprofen; and

FIG. 10 is a graphical view of the cytotoxicity of doxycycline, flurbiprofen and combinations thereof.

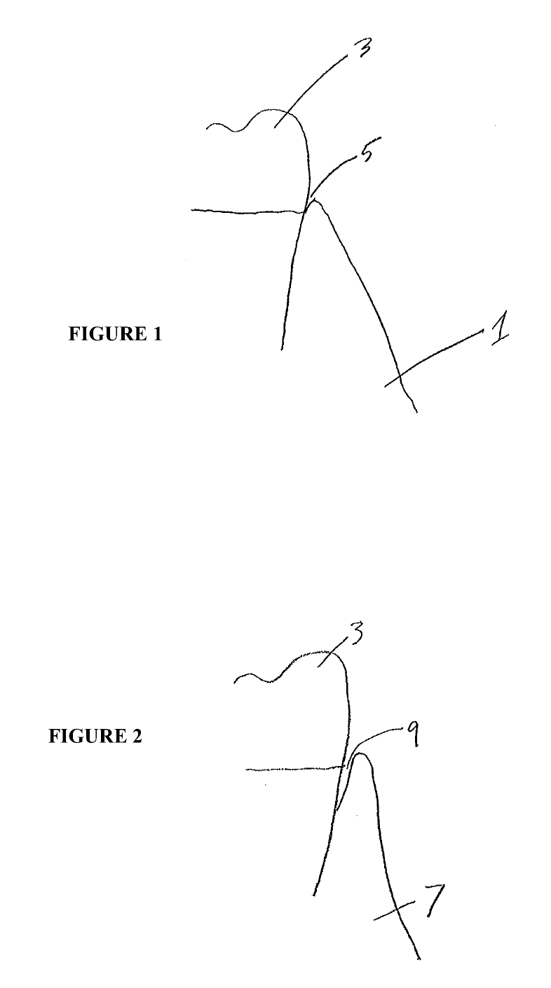

### DETAILED DESCRIPTION OF THE INVENTION

The methods and drug delivery technology of the present invention are improvements over traditional surgical and non-surgical procedures, which can be painful, costly and may not be effective. The improvements include, inter alia, a minimally-invasive method of treating chronic periodontitis and other oral diseases which is safe and effective. In one embodiment, treatment of chronic periodontis (CP) in a subject begins with a diagnosis of CP for the subject, then administering a plurality of microcapsules comprised of one medication or a combination of medications, to the desired site in the periodontal pocket or other affected area. The affected area can include the oral cavity, tongue, oral mucosa, teeth themselves, prosthetic devices such as dentures and combinations thereof. The subject can be a human subject. The subject may also be a cat, dog, pig, cow, horse or any other mammal. The microcapsules contain one or more pharmacologic agents, the one or more pharmacologic agents being stored in at least one nano-compartment of the microcapsule. As used herein, “nano” refers to nanoparticles and processes wherein the scale is in the range of 1 nanometer (nm) to 1 micrometer (μm). As used herein, “micro” refers to particles and processes in the range of 1 μm to 1000 μm. When it is not necessary to distinguish between nano- and micro-sizes, the term “micro” is used generically to refer to objects in both the nano- and micro-size ranges. By a plurality of microcapsules is meant a large number of microcapsules. In one embodiment, these microcapsules can be nanospheres. A typical microcapsule will range from 100 nm-1,000 nm, an average sized microcapsule would be about 5×10⁻⁷ m, or 2.5×10⁻⁷ in radius. Hence the microcapsule volume would be about 1.6×10⁻²° m³*π, or about 4.9×10⁻²⁰ m³ per average microcapsule. The number of microcapsules contained in an average administration would be about 2×10¹² or 200 billion. Depending on the size of microcapsule used, and the volume of microcapsules administered, the number of microcapsules in a single application or dose would range from a few thousand to several hundred billion. For example, a single application or dose could range from about 1,000 to about 400 billion microcapsules, from about 1,000 to about 100,000 microcapsules, about 100,000 to about 1,000,000 microcapsules, about 1,000,000 to about 100,000,000 microcapsules, about 100,000,000 to about 1 billion microcapsules, about 1 billion to about 100 billion microcapsules, about 100 billion to about 200 billion microcapsules or about 200 billion to about 400 billion microcapsules.

The microcapsules may be made in any suitable manner. For example, the microcapsules can be made from a master and a photodynamic process wherein light is shone through the master on a silicon wafer resulting in the creation of uniformly sized wells with one or more nano-compartments. PLGA (polylacticglycolic acid) is then placed over the wafer, filling the wells (in a manner similar to microchip manufacturing). Next, a gelatin mold is made from the PLGA template creating a duplicate of the silicone. Each of the nano-compartments in the gelatin mold is then filled with the pharmacologic agents and PLGA. The gelatin wafer is then dissolved in water, leaving the PLGA-filled microcapsules that contain the pharmacologic agents.

The size and the shape and material of the microcapsules control the release characteristics. One advantage of this technique is that the release characteristics of the resulting microcapsules can be controlled and specifically designed

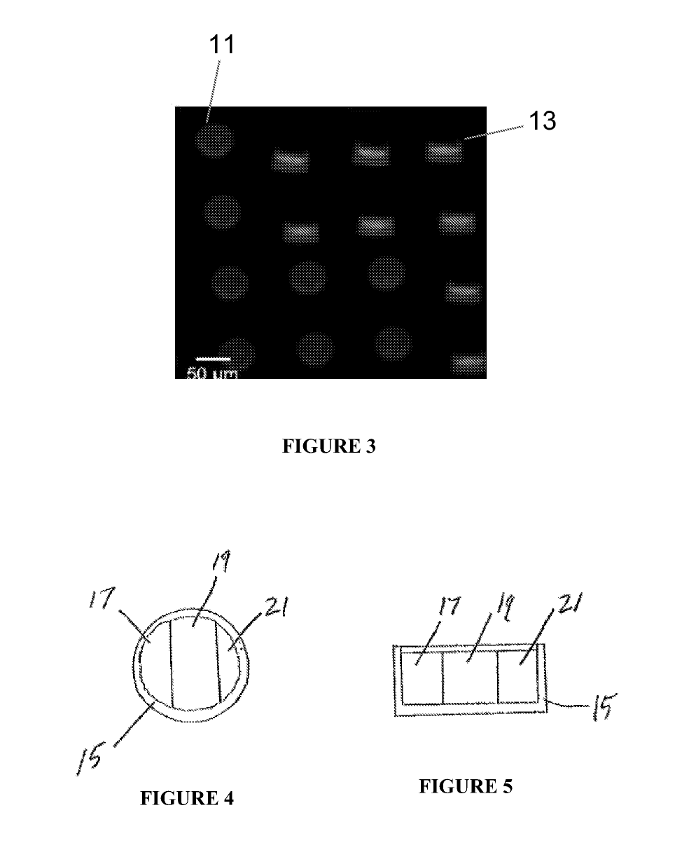

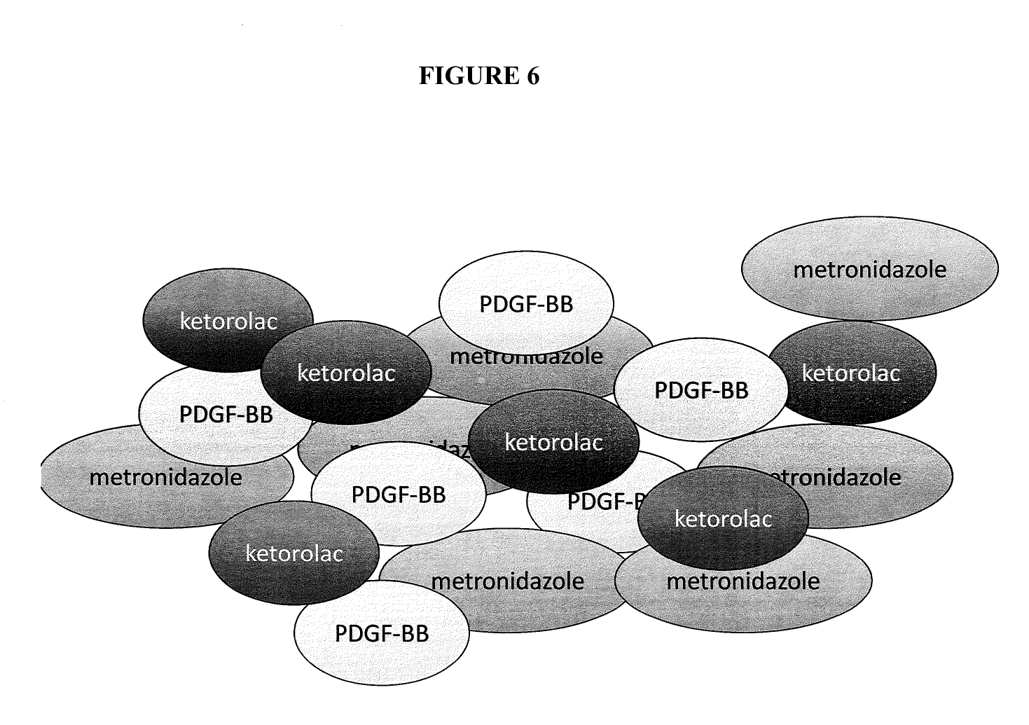

This method is further described in Provisional Patent 61/267,612, and International Application PCT/US2008/011260, the disclosures of which are incorporated herein by reference. The shape of the microcapsules can be varied. For example, the microcapsules can be formed in any conventional shape, including, for example, cylindrical, rectangular, spherical, diamond shape, oblong or triangular. In one embodiment the microcapsules are nanospheres. The size of the microcapsules may also be varied. For example, the microcapsules may be in the range of about 100 nm to about 1,000 μm. An example of microcapsules can be seen in FIG. 3. FIG. 3 represents an exemplary, cylindrical microcapsule with a size of about 50 μm. A top view of a microcapsule 11 shows the top polymer layer of the sealed microcapsule. A sideview of a microcapsule 13 shows the pharmacologic material as the lighter in color region surrounded by the microcapsule structure as the slightly darker region. FIGS. 4 and 5 represents two more views of an exemplary microcapsule 15. FIG. 4 is a top view of exemplary microcapsule 15 showing a first microcapsule 17, a second microcapsule 19 and a third microcapsule 21. FIG. 5 is a side view of exemplary microcapsule 15 showing a first microcapsule 17, second microcapsule 19 and a third microcapsule 21. In other embodiments, more or less microcapsules may be included in each matrix, as shown in FIG. 6, and further described below.

The microcapsules are formed of materials that are designed to have known degradation rates once introduced to a subject's body. Furthermore, each microcapsule can contain one or more nano-compartments, which contain different pharmacological agents. These different pharmacological agents may be contained in a PLGA matrix, as shown in FIG. 6, or in an administration vehicle such as a paste, a rinse, an ointment, a salve, a suppository, a chip a gel, a syrup, a foam, a matrix and combinations thereof, that is designed to degrade in a controlled temporal fashion. These microcapsules can be formed of different thicknesses and different materials so that the contents of each of the microcapsules are released in a manner determined in advance per the treatment design and so as to be optimal for the given application. For example, a microcapsule designed to degrade and release its contents quickly could have a wall thickness of about 10 nm. As another example, a microcapsule designed to degrade and release its contents more slowly could have a wall thickness of about 20 nm.

In one embodiment, the microcapsules are formed of a polylactic co-glycolic acid (PLGA) polymer of varying thicknesses. The microcapsules may also be formed of poly (lactic acid) (PLA), poly (glycolic acid) (PGA), poly (ε-caprolactone) and other biodegradable polymers. The one or more microcapsules may also be formed of PLGA, or other suitable biodegradable polymers, of varying thicknesses and size. In one embodiment, a matrix may be designed and administered which has one nanosphere which will release the pharmaceutical agent or products upon hydrolysis of the PLGA structure. In another embodiment, the matrix is designed and administered with more than one microcapsule and one or more than one pharmaceutical agent(s). This combination microcapsule is designed to release the pharmaceutical agent or agent(s) contained within it after the release of the pharmaceutical agent or agent(s) from a second microcapsule.

Referring back to FIG. 3, FIG. 3 is representative of an embodiment of a matrix with one microcapsule containing at least one pharmaceutical agent. The plurality of microcapsules (the matrix) may contain a combination of two or more pharmaceutical agents. When the matrix of microcapsules comprises two or more pharmaceutical agents, a first pharmaceutical agent may be at least one anti-microbial agent, a second pharmaceutical agent may be a non-steroidal anti-inflammatory drug (NSAID) and a third pharmaceutical agent may be a growth factor, all of which will be released from the microcapsules upon hydrolysis of the structural polymers and in a pre-determined controlled manner. Other agents may also be stored in the microcapsule and released including anti-tumor necrosis factors (TNF), cytokine inhibitors, matrix metalloproteinase (MMP) inhibitors, bisphosphonates, botanical products, herbal products, curcumins, other polyphenols and combinations of these products, among others. Other pharmaceutical agents stored in the microcapsule can include flavoring agents, fluoride, breath freshening agents, anti-bacterial agents, whitening agents, analgesic agents and combinations thereof.

The pharmaceutical agents stored within the microcapsules and the nano-compartments of the microcapsules can be in any suitable form. For example, the pharmaceutical agents can be in a powder, paste, foam, particulate or liquid form. As another example, the pharmaceutical agents can be incorporated into a chip or wafer before placement within the microcapsules or the nano-compartments of the microcapsules.

In another embodiment, a microcapsule may be designed with two or more separate microcapsules housed within the microcapsule matrix. The two or more separate microcapsules can be designed to release their contents of one or more pharmaceutical agents at different times than each other. For example, “different times” is understood in connection with the present disclosure to mean, sequentially or staggered release with a period of time between the release of the pharmaceutical agents. The period of time can be from seconds to minutes to hours. As an example, one microcapsule can have a relatively thinner coating of PLGA, or other composition of PLGA, or other suitable biodegradable polymer that can hydrolyze and release its contents, before the hydrolysis and release of the contents of a second and third microcapsule with relatively thicker coatings of PLGA, or other suitable biodegradable polymer.

FIG. 6 is a representative view of a matrix containing a plurality of microcapsules containing different agents, with each microcapsule being diagrammatically represented by an oval. In the embodiment shown in FIG. 6, many of the microcapsules contain a different pharmacologic agent. The matrix, which includes microcapsules containing different pharmacologic agents, can be made so that the release of the different materials is designed to effect different results at different times. In the embodiment shown in FIG. 6, the metronidazloe microcapsules will release during a 7-14 day period, the ketorolac will release over a 7-28 day period and the PDGF-BB will release over a 4-12 week period. This embodiment has been designed to effect wound healing over about a 12 week period.

This design choice, having the ability to deliver a first pharmaceutical agent contained in a first microcapsule at a specified time followed by the delivery of a second pharmaceutical agent contained in a second microcapsule and a third pharmaceutical agent contained in a third microcapsule, allows the customization of a pharmacologic delivery regimen. Another design choice, having the ability to deliver a first pharmaceutical agent contained in a first nano-compartment of a microcapsule at a specified time followed by the delivery of a second pharmaceutical agent contained in a second nano-compartment of the same microcapsule and the delivery of a third pharmaceutical agent from a third nano-compartment of the same microcapsule also allows the customization of a pharmacologic delivery regimen.

In one embodiment, a first microcapsule, or a first nano-compartment of a microcapsule, a second microcapsule, or a second nano-compartment of a microcapsule and a third microcapsule, or third nano-compartment of a microcapsule can be designed to hydrolyze and release a stored pharmaceutical agent at the same time. In this embodiment the three pharmaceutical agents may be the same or they may be different. Further, in this embodiment, if the pharmaceutical agents are different, the pharmaceutical agents may be chosen for their synergistic effect. In other embodiments, there may be four or more microcapsules or nano-compartments.

In another embodiment, a first microcapsule, or a first nano-compartment of a microcapsule, can be designed to hydrolyze and release a stored pharmaceutical agent at a later time than a release from a second microcapsule, or a second nano-compartment of a microcapsule, and third microcapsule or a third nano-compartment of a microcapsule. In this embodiment, the pharmaceutical agents may be the same or they may be different. Further, in this embodiment, if the pharmaceutical agents are different, the pharmaceutical agent stored in the first microcapsule can be designed to be released at a desired time after the release of a second pharmaceutical agent from the second microcapsule and before the release of a third pharmaceutical agent from the third microcapsule. In this embodiment, the timed release of different pharmaceutical agents can be designed to more effectively treat a diagnosed disease.

In one example, a first microcapsule can contain at least one non-steroidal anti-inflammatory drug (NSAID), a second microcapsule can contain at least one anti-microbial agent, a third microcapsule can contain at least one growth factor and a fourth microcapsule can contain anti-(TNF), cytokine inhibitors, MMP inhibitors, bisphosphonates, botanical products, herbal products, curcumins and combinations of these products, among others. The NSAID stored in the first microcapsule can be selected from ketorolac tromethamine, ibuprofen, naproxen, fenoprofen, ketoprofen, flurbiprofen, oxaprozin, indomethacin, sulindac, etodolac, diclofenac, piroxicam, meloxicam, tenoxicam, droxicam, lornoxicam, isoxicam, mefenamic acid, meclofenamic acid, flufenamic acid, tolfenamic acid, celecoxib, diflunisal, meclofenamate sodium, and COX-2 selective inhibitors including meloxicam, valdecoxib, colecoxib and rofecoxib, among others. The anti-microbial agent stored in the second microcapsule can be selected from metronidazole, penicillins, cephalosporins, quinolones, sulfonamides, aminoglycosides, macrolides, tetracyclines, doxycycline, minocycline, erythromycin, Pen G, Pen VK, ampicillin, amoxicillin, augmentin, cephalexin, ciprofloxacin, azithromycin, clindamycin, vancomycin, cyclic lipopeptides, glycylcyclines, oxazolidinones, quaternary ammonium compounds, boric acid, brilliant green, chlorhexidine gluconate, iodine, hydrogen peroxide, phenol compounds, among others.

In one example, the first microcapsule can contain a combination of an anti-microbial agent and an NSAID. For example, the first microcapsule can contain doxycycline and flurbiprofen. Unexpectedly, the present description demonstrates that the combination of these two compounds at appropriate concentrations does not increase cytotoxicity and can be effective at reducing inflammation upon administration. The appropriate concentration of low doses of anti-microbials is understood to include sub-anti-microbial doses.

The anti-microbial(s) may be delivered in a dose that is sub-antimicrobial so as to down-regulate the inflammatory host response characteristics through inhibition of multiple tissue destructive proteinases and cytokines, including matrix metalloproteinase (MPP-8, MPP-13, MMP-9), interleukin 1β, and tumor necrosis factor α, without maintaining a high enough concentration to kill or inhibit the growth of microorganisms.

The growth factor stored in the third microcapsule can be selected from platelet derived growth factor, autocrine motility factor, bone morphogenetic proteins including BMP-2, autocrine motility factor, epidermal growth factor, erythropoietin, fibroblast growth factor, granulocyte-colony stimulating factor, granulocyte-macrophage colony stimulating factor, growth differentiation factor-9, hepatocyte growth factor, hepatoma derived growth factor, insulin-like growth factor, migration-stimulating factor, myostatin, nerve growth factor, thrombopoietin, transforming growth factors alpha and beta, vascular endothelial growth factor, placental growth factor, foetal bovine somatotrophin, IL-1-, IL-2-, IL-3, IL-4, IL-5, IL-6 and IL-7 among others.

The first, second, third and fourth microcapsules can be designed with varying amounts of PLGA or other suitable biodegradable polymers, which hydrolyze and release their contents at different times. For example, if the second microcapsule has been designed to release its contents first, the second microcapsule will have a relatively thinner coating of PLGA or other suitable biodegradable polymer as compared to a first microcapsule. The PLGA or other suitable biodegradable polymer is hydrolyzed in the oral cavity, releasing the contents of the respective microcapsule. The relatively thinner the coating of PLGA or other suitable biodegradable polymers and the smaller the microcapsule, the more quickly the coating is hydrolyzed and the more quickly the contents of the microcapsule are released.

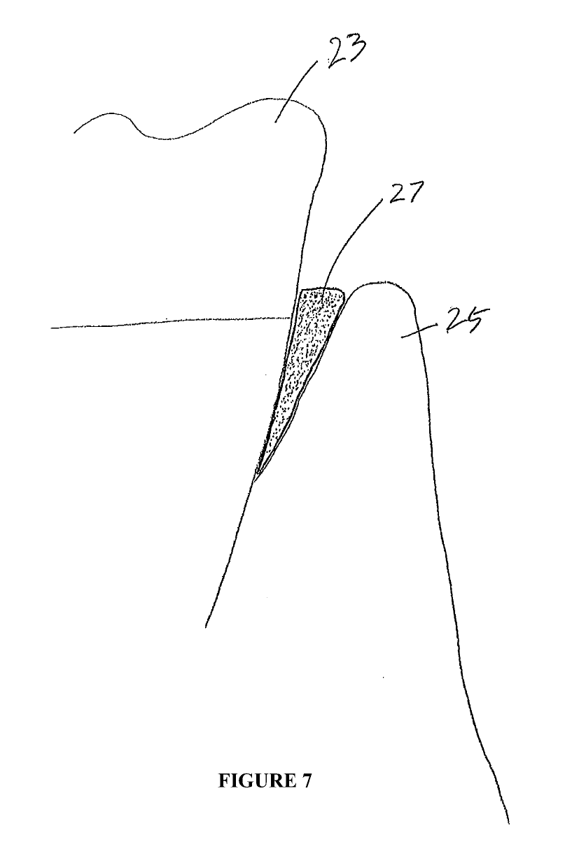

The pharmaceutical agents stored in the microcapsules can be chosen upon a diagnosis of an oral disease in a subject or upon the determination of a subject in need of oral therapy. In one embodiment, the subject is diagnosed with periodontal disease. Upon a diagnosis of periodontal disease, a plurality of microcapsules can be dispersed in any suitable form of administration vehicle, for example an aqueous solution, a rinse, an ointment, a salve, a suppository, a chip a paste, a gel, a foam, a syrup, a matrix of microcapsules and combinations thereof. The carrier and the plurality of microcapsules can then be applied to an affected area, such as into a pocket as shown in FIG. 7, where a subject's tooth 23 and a subject's gum tissue 25 have detached. The affected area can also include the oral cavity, tongue, oral mucosa, teeth themselves, prosthetic devices such as dentures and combinations thereof. In the pocket created between tooth 23 and gum tissue 25, a dispersal of a plurality of microcapsules 27 have been placed. The dispersal of a plurality of microcapsules 27 can be administered one time for an extended period of delivery of different pharmaceutical agents. This may avoid the need for continual applications, although continual or periodic applications are contemplated by the present invention, as well. By “continual or periodic applications” is meant hourly, daily, weekly, or monthly applications either performed during professional appointments or performed by a user themselves. The applications can be mixed and designed just prior to application, or they may be attained in a pre-mixed format based on the perceived need. One advantage to applying a dispersal of a plurality of microcapsules as a local delivery is that the pharmaceutical agent(s) stored in the dispersal of a plurality of microcapsules produces fewer toxic side effects than systemically delivered drugs. A plurality of microcapsules can also be administered to a subject who has been diagnosed with a periodontal disease, such as periodontitis, CP, peri-implantitis, sinus infections, mucositis, dental carries, gingivitis and halitosis. In another embodiment, it is determined that a subject is in need of oral therapy. Upon a determination that a subject is in need of oral therapy, a plurality of microcapsules can be administered to the subject. The plurality of microcapsules can be administered to treat a disease, heal an oral wound which could be caused by an extraction or a sinus augmentation, or guide bone regeneration guide tissue regeneration or both.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

### Example 1

In the following example, one embodiment of a method of treating an oral disease in a subject is described.

A human subject is diagnosed with periodontal disease by a qualified dentist. The dentist determines that the person has periodontal disease, by among other indicators, measuring the depth of periodontal pockets formed between the person's tooth and gum tissue. Upon this diagnosis, the dentist, or other qualified health care provider, makes a choice, based on the individual subject, the severity and extent of the disease, about what treatment regimen to take. In this example, the dentist determines that the person's periodontal pockets are about 5 mm deep, and decides to treat the subject with an administration of a plurality of microcapsules.

The dentist chooses, among several design options of microcapsules, to administer a plurality of materials that each have three different nano-compartments. Each of the three nano-compartments will hold a different pharmaceutical agent. The first nano-compartment will hold an anti-microbial, in this example metronidazole. The second nano-compartment will hold a non-steroidal anti-inflammatory drug (NSAID), in this example ketorolac tromethamine. The third nano-compartment will hold a growth factor, in this example a platelet derived growth factor (PDGF), or anti-collagenase.

The microcapsules will be formed of polylactic co-glycolic acid (PLGA) with each nano-compartment designed to release its contents at different times. The first nano-compartment can be designed to release first, within hours to days of being exposed to the oral cavity. The second nano-compartment can be designed to release second, within a few days to several days upon being exposed to the oral cavity. The third nano-compartment can be designed to release third, within several weeks upon being exposed to the oral cavity. The dentist chooses a microcapsule with these specific nano-compartments and pharmaceutical agents to first reduce the bacterial or microbe concentration in the local area surrounding the affected area by releasing the anti-microbial metronidazole, followed by the release of an anti-inflammatory such as ketorolac tromethamine, which is then followed by a growth factor such as PDGF to aid in growth of the gum tissue and tooth structure.

The plurality of microcapsules would be delivered to the dentist dispersed in an aqueous solution, a paste, a matrix, a foam, a syrup, a gel, a rinse, an ointment, a salve, a suppository, a chip, and combinations thereof. The dentist would then take the dispersal of the plurality of microcapsules and apply it to the subject's periodontal pocket, substantially filling it. The dentist would apply the dispersal of the plurality of microcapsules into the subject's periodontal pocket in any suitable way, including injecting the dispersal into the open pocket with a syringe, using a tool, for instance a small spatula type tool, to pack the dispersal into each pocket, or inserting a bolus of a predetermined quantity of the dispersal of the plurality of microcapsules which has been preformed to keep a desired shape. The dispersal is designed to remain in the subject's periodontal pocket for an extended period, in this example, several days. While the dispersal is in the subject's periodontal pocket, the PLGA structures of the microcapsules and nano-compartments are continually hydrolyzed, and will eventually release their contents to the local tissue surrounding the pocket.

### Example 2

In the following example, one embodiment of a method of treating an oral disease in a subject is described.

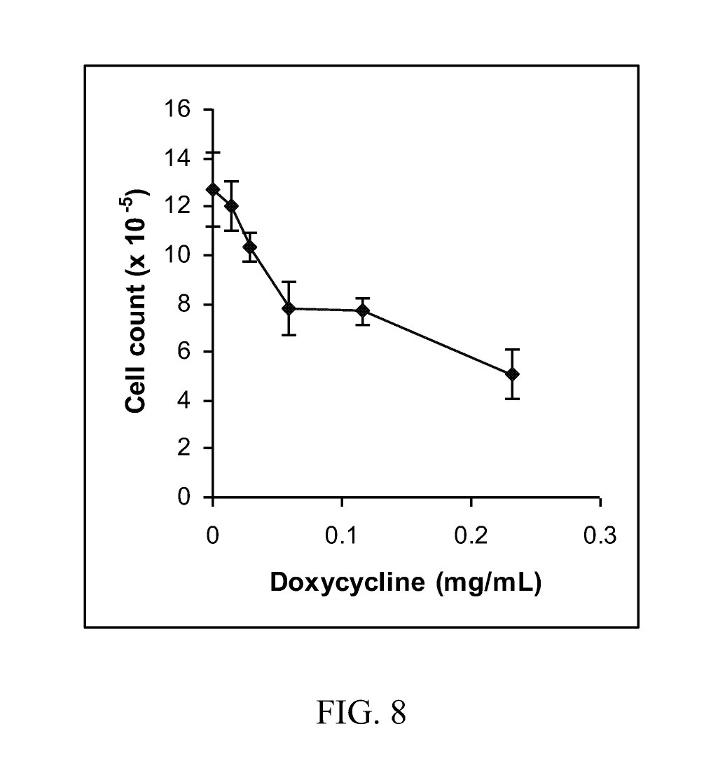

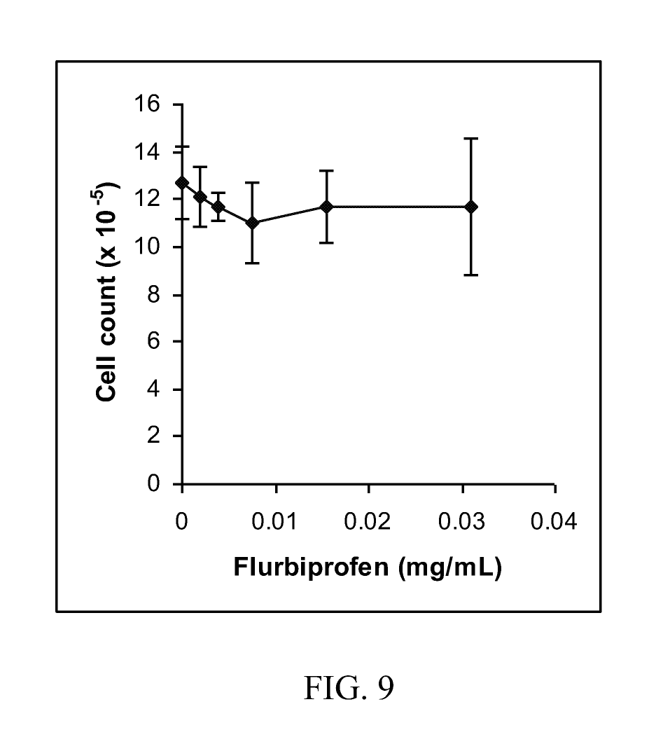

This example is similar to Example 1, with one difference being that the dentist chooses to administer a plurality of microcapsules wherein one nano-compartment holds the NSAID flurbiprofen and another nano-compartment holds the anti-microbial doxycycline. Doxycycline has a relative cytotoxicity of 0-0.231 mg/mL using gingival fibroblasts as the test cells. These results are shown in FIG. 8. Flurbiprofen has a relative cytotoxicity of 0-0.031 mg/mL using gingival fibroblasts as the test cells. To determine whether the co-administration of these two compounds was cytotoxic, the following experiments were carried out.

Since flurbiprofen had a relatively low cytotoxicity, a concentration of 0.031 mg/mL was used in this experiment. Since doxycycline indicated a relatively increasing cytotoxicity with increasing concentration, two concentrations were used, 0.014 mg/mL and 0.116 mg/mL. The 0.116 mg/mL concentration of doxycycline is equivalent to about 0.012 mg/mL in serum. Fourth-fifth passage gingival fibroblasts were plated at 5*104 cells/p100 in Dulbecco's modified Eagle's medium (DMEM) and 10% Fetal Bovine Serum (FBS). Twenty four hours after plating, the base medium was replaced with the test medium in triplicate. Cultures were fed every other day and harvested about 5 days later.

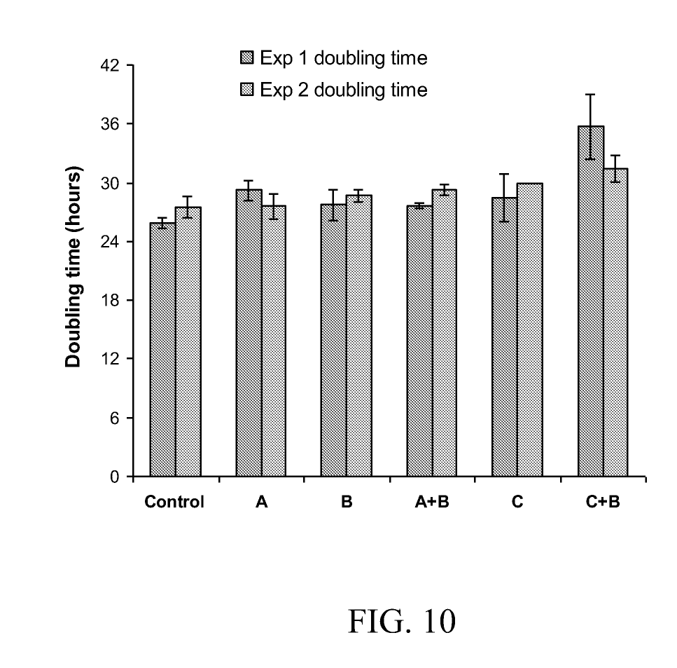

Cell counts were obtained and population doubling times (PDT) were calculated as follows: PDT=[24 hours/day]*[days]*[number of doublings]. The number of doublings=(LN[cell output)−LN(plating efficiency*input)/LN(2). The results are shown in FIG. 10, with the data presented as the mean (SD) for each experiment.

In FIG. 10, A is 0.014 mg/mL doxycycline, B is 0.031 mg/mL flurbiprofen and C is 0.116 mg/mL doxycycline. As can be seen in FIG. 10, the addition of flurbiprofen to the lower dose of doxycycline does not significantly increase the cytotoxicity of the combination. Compared to growth with doxycycline alone, no significant change in population doubling time was observed when the combination of flurbiprofen and the lower dosage of doxycycline is exposed to cells. As can also be seen from FIG. 10, the combination of flurbiprofen and the higher dose of doxycycline increase the population time by about 5-26%.

What is claimed is:

1. A method of treating an oral disease in a subject comprising:
diagnosing the subject; and
administering a plurality of microcapsules, the microcapsules comprising at least one pharmaceutical agent in at least one nano-compartment of the microcapsule.

2. The method of claim 1, wherein the at least one nano-compartment comprises at least a first pharmaceutical agent and a second pharmaceutical agent.

3. The method of claim 2, wherein the first pharmaceutical agent is an anti-microbial agent.

4. The method of claim 2, wherein the second pharmaceutical agent is a non-steroidal anti-inflammatory drug (NSAID).

5. The method of claim 2, further comprising a third pharmaceutical agent.

6. The method of claim 5, wherein the third pharmaceutical agent is a growth factor.

7. The method of claim 1, wherein the microcapsule comprises two or more nano-compartments.

8. The method of claim 7, wherein a first nano-compartment releases a pharmaceutical agent prior to the release of a pharmaceutical agent contained in a second nano-compartment.

9. The method of claim 8, wherein the first nano-compartment contains a first pharmaceutical agent and the second nano-compartment contains a second pharmaceutical agent.

10. The method of claim 9, wherein the first pharmaceutical agent is an anti-microbial agent.

11. The method of claim 9, wherein the second pharmaceutical agent is a non-steroidal anti-inflammatory drug (NSAID).

12. The method of claim 8, further comprising a third microcapsule which releases a third pharmaceutical agent.

13. The method of claim 12, wherein the third pharmaceutical agent is a growth factor.

14. The method of claim 1, wherein the oral disease is selected from the group consisting of peri-implantitis, sinus infections, mucositis, dental carries, gingivitis, halitosis and periodontitis.

15. The method of claim 1, wherein the plurality of microcapsules are administered to an area affected by the disease.

16. The method of claim 15, wherein the area affected by the disease is a pocket formed between a gum tissue and a tooth of the patient.

17. The method of claim 15, wherein the area affected by the disease is selected from the group consisting of the oral cavity, tongue, oral mucosa, teeth, prosthetic devices and combinations thereof.

18. The method of claim 1, where the subject is human.

19. The method of claim 1, wherein the nano-compartment comprises polylactic co-glycolic acid (PLGA).

20. The method of claim 1, wherein the microcapsule is about 100 nm to about 1,000 μm in size.

21. An oral therapy comprising:
determining a subject in need of the oral therapy; and
administering a plurality of microcapsules, the microcapsules comprising at least one pharmaceutical agent in at least one nano-compartment of the microcapsule.

22. The therapy of claim 21, wherein the therapy is for the treatment of a disease.

23. The therapy of claim 21, wherein the therapy is for the healing of an oral wound.

24. The therapy of claim 23, wherein the wound is an extraction wound, a sinus augmentation wound, or both.

25. The therapy of claim 21, wherein the therapy is for guided bone regeneration, guided tissue regeneration or both.

26. The method of claim 1, wherein the plurality of microcapsules is about 1,000 to about 400 billion microcapsules.

27. The method of claim 1, wherein the plurality of microcapsules is about 1,000 to about 100,000 microcapsules.

28. The method of claim 1, wherein the plurality of microcapsules is about 100,000 to about 1,000,000 microcapsules.

29. The method of claim 1, wherein the plurality of microcapsules is about 1,000,000 to about 100,000,000 microcapsules.

30. The method of claim 1, wherein the plurality of microcapsules is about 100,000,000 to about 1 billion microcapsules.

31. The method of claim 1, wherein the plurality of microcapsules is about 1 billion to about 100 billion microcapsules.

32. The method of claim 1, wherein the plurality of microcapsules is about 100 billion to about 200 billion microcapsules.

33. The method of claim 1, wherein the plurality of microcapsules is about 200 billion to about 400 billion microcapsules.

34. The method of claim 1, wherein the plurality of microcapsules are contained in an administration vehicle, the administration vehicle selected from the group consisting of a paste, a rinse, an ointment, a salve, a suppository, a chip, a gel, a foam, a syrup, a matrix and combinations thereof, for administration.

35. The method of claim 1, wherein the at least one pharmaceutical agent is selected from the group consisting of a flavoring agent, fluoride, breath freshening agent, anti-bacterial agent, anti-inflammatory agent, whitening agent, analgesic agent and combinations thereof.
